(19) United States
(12) Patent Application Publication
(10) Pub. No.: US 2024/0269288 A1
(43) Pub. Date: Aug. 15, 2024

(54) NON-CONTACT LITHOTRIPSY USING PHOTONIC NANOPARTICLES

(71) Applicant: The Cleveland Clinic Foundation, Cleveland, OH (US)

(72) Inventors: Vijay KRISHNA, Beachwood, OH (US); Smita DE, Bratenahl, OH (US); Ian Thomas HOULIHAN, Olmsted Township, OH (US)

(21) Appl. No.: 18/566,802

(22) PCT Filed: Jun. 6, 2022
(86) PCT No.: PCT/US2022/032336

§ 371 Date: Dec. 4, 2023

Related U.S. Application Data
(60) Provisional application No. 63/196,938, filed on Jun. 4, 2021.

(51) Int. Cl. A61K 41/00; A61B 18/26; A61K 9/51

(52) U.S. Cl. CPC A61K 41/0028; A61B 18/26; A61K 9/5123; A61B 2018/263

(57) ABSTRACT

Methods of non-contact lithotripsy are disclosed, wherein photonic nanoparticles are delivered in the vicinity of kidney stones, e.g. within a kidney. The nanoparticles then are irradiated with non-ionizing electromagnetic radiation to activate the nanoparticles and fragment the stone. In preferred embodiments, the nanoparticles are placed effectively into contact with the kidney stone, via functionalizing the particles with ligands that will adhere to the known or anticipated chemical composition of the stone surface. A combination of a kidney stone and a photonic nanoparticle in its vicinity or adhered thereto at its surface also is disclosed.

### BACKGROUND

The prevalence of nephrolithiasis has been rising over the last several decades with approximately 1 in 10 individuals affected in the United States. The disease is associated with significant morbidity including pain, infection, and renal insufficiency, as well as costs, which were estimated to be over $5 billion in 2000. For these reasons, efficient and effective surgical management is critical for patients whose stones do not pass spontaneously. Current treatment options for stones include ureteroscopy, extracorporeal shock wave lithotripsy (ESWL), percutaneous nephrolithotomy, and rarely laparoscopic or open surgery. Unfortunately, more than 40% of patients may not be stone free after surgery and residual stones can have severe consequences. Furthermore, there is an estimated 50% recurrence rate such that patients often need multiple procedures over a lifetime, which can require repeated exposure to ionizing radiation and anesthesia, both of which have associated risks. Given the above, it is desirable to explore novel technologies to improve stone free rates as well as reduce radiation exposure from fluoroscopy during stone procedures.

Currently, ureteroscopy is the most common form of surgical treatment for stones. This requires visualization of the stone(s) with a ureteroscope followed by laser lithotripsy, or fragmentation or dusting of the stone(s) using a contact laser (i.e. the laser emitter must physically contact the stone in order to be effective at breaking it up). Much time can be spent chasing around stones and stone fragments in order to get close enough to adequately fragment them with the contact lasers. Worse, there are cases in which the stones can be seen but not touched by the laser fiber due to their location and/or limitations in the flexibility or maneuverability of the endoscope. In addition, these lasers generate significant heat and there is concern for potential thermal damage during laser lithotripsy. Also, ureteroscopy generally requires fluoroscopic guidance thus exposing the operating team and patient to radiation in most, if not all, cases.

In addition to the foregoing limitations, ureteroscopy may not effectively treat large stones (e.g. >2 mm dia.) because the size of the endoscope used in such procedures is limited by the diameter of the ureter through which it must be inserted. Current practice is to place a ureteral stent between the kidney and the bladder at the end of the surgery to maintain ureteral patency. This both combats post-treatment swelling of the ureter and facilitates passage of any residual stone fragments. However, such stents yield their own morbidities, not the least being patient pain and discomfort. A large proportion of patients report that dealing with the ureteral stent after ureteroscopy is worse than the procedure itself and materially diminishes quality of life while in place—which can be up to several weeks.

Another common treatment is ESWL, which has the merits of being non-invasive. However, its utility is severely limited by stone location and size, two factors that to a lesser, but important extent also can limit ureteroscopy effectiveness. Stone quality (i.e. composition) is another important limitation on the utility of ESWL, as is (very importantly) patient body habitus. ESWL does not work well in larger patients in whom the shockwaves must travel further, a key consideration given the rise in obesity. ESWL also can result in damage to surrounding tissues. For example, because the acoustic shockwave is introduced from outside the body, to reach the stone it usually must travel through critical organs, resulting in a potential for damage. Moreover, ESWL also requires fluoroscopy, which it is desirable to minimize. Further still, if the small fragments resulting from the shockwaves collect and become packed within the ureter as they are trying to pass, they can block the ureter requiring emergency intervention, Finally, ESWL is contraindicated in many patients, such as those being treated with anticoagulants for various conditions, because the shockwaves may produce kidney hemorrhage.

### SUMMARY

Methods are disclosed to perform lithotripsy by delivering photonic nanoparticles in the vicinity of a kidney stone within a patient, and thereafter activating the nanoparticles with non-ionizing electromagnetic radiation to thereby fragment the kidney stone.

A combination of a kidney stone and a photonic nanoparticle at a surface of the kidney stone also is disclosed.

A kidney stone in contact with a photonic nanoparticle at its surface also is disclosed.

### BRIEF SUMMARY OF FIGURES

FIG. 1 shows comparative images of kidney stones treated (or not) in vitro with photonic nanoparticles disclosed herein according to a first example, pre- and post-irradiation of the stones.

FIG. 2 shows comparative images of kidney stones treated in vitro with photonic nanoparticles disclosed herein according to a second example, pre- and post-irradiation of the stones.

FIG. 3 shows comparative pre- and post-irradiation micro-CT images of kidney stones treated in vitro with photonic nanoparticles disclosed herein according to a third example.

FIG. 4 plots the decrease in Hounsfield units and corresponding increase in specific surface area for the kidney stones treated in the third example.

FIG. 5 shows comparative images of kidney stones treated in vitro with photonic nanoparticles disclosed herein according to a fourth example, pre- and post-irradiation of the stones.

FIG. 6 shows comparative images of kidney stones treated in vitro with photonic nanoparticles disclosed herein according to a fifth example, pre- and post-irradiation of the stones.

### DESCRIPTION

Disclosed here is a method of treating kidney stones, potentially of any size, in a manner that does not require physical contact between the treating instrument (cf. a conventional contact laser) and the stone. Broadly speaking, the novel methodology includes two stages. The first is placing photonic nanoparticles in the vicinity of, preferably into physical contact with, the stone. The second is applying non-ionizing electromagnetic radiation energy (such as infrared radiation, which can be supplied by a laser operating within an IR wavelength) to activate the nanoparticles. Activation of the nanoparticles generates vibrational (acoustic shockwave) and/or thermal energy that fragments the stone.

### Photonic Nanoparticles

The photonic nanoparticles are nano-scale particles that can generate mechanical, chemical or thermal energy, or a combination thereof, upon irradiation with electromagnetic (i.e. light) energy. Preferred photonic nanoparticles in the instant application will be effective to absorb infrared (IR) energy, preferably within a range of 750 to 3000 nm wavelength, and to convert at least a portion of the absorbed energy to vibrational (mechanical) energy that can create heat or sound. That vibrational energy can perform work applied to an adjacent or contacting kidney stone to thereby fragment the stone as a mode of treatment thereof.

In a first embodiment, preferred photonic nanoparticles are fullerenes, which represent an allotrope of carbon wherein individual carbon atoms are arranged in a mesh network of closed-carbon rings of five to typically seven atoms. Fullerenes are known generally, and may constitute closed-mesh molecules of carbon atoms that can be spherical, ellipsoid, tubular, as well as other closed-form shapes. Open-mesh fullerenes, such as flat-sheet molecules formed from a carbon-mesh structure, such as graphene, also are known and may be suitable. Importantly, fullerenes can be activated to generate acoustic shockwaves via focused radiation energy across a wide spectrum, including throughout the aforementioned IR range. This enables the use of lasers (that deliver the particle-activation energy) operating at wavelengths that will not be well absorbed by water or human tissues, which therefore will not generate substantial heat that can damage those tissues (unlike conventional thulium fiber and holmium lasers).

For example, near-IR irradiation may be supplied to activate fullerene nanoparticles via relatively low-energy photons delivered from lasers operating at low power within an established ‘biological transparency window’ in the near-IR spectrum. Currently known IR biological transparency windows include the following: 700-950 nm, 1000-1350 nm, 1600-1870 nm and 2100-2300 nm. These windows represent IR wavelengths to which human soft tissues have been shown to be substantially transparent to irradiation—meaning those tissues (represented largely by the water they comprise) will not absorb significant energy that may damage the tissues from irradiation at such wavelengths. The wavelength of IR radiation should be selected to minimize water—(and therefore tissue) absorption of IR energy, thus reducing or minimizing the resultant generation of heat. Wavelengths within the aforementioned biological windows are believed to satisfy this criterion, and a laser of 785 nm wavelength operating at <1 W has been shown to be effective in laboratory testing. It is contemplated such a system will be effective for retrograde energy delivery. If delivered transcutaneously, higher power (e.g. <5 W) may be appropriate in order to penetrate intervening tissues before the energy arrives at the treatment site.

The delivered IR radiation is absorbed by and activates the fullerene nanoparticles. It is not relied on to directly fragment solid-mineral kidney stones. For this reason, far lower power than current conventional practice can be used; e.g. from 0.1 to 5 W (preferably 0.5 to 2 W, more preferably not more than 1 W for retrograde energy delivery). Low-energy photons within a low-absorptivity biological window are unlikely to damage surrounding tissues and will not generate significant heat in those tissues, owing to their low power and the fact that water molecules do not absorb energy well in such windows. Moreover, for transcutaneous energy delivery (also discussed below), the proposed infrared wavelengths generally are not harmful to the skin, at least during such relatively short exposure as would occur to facilitate treatment of kidney stones.

Among fullerenes, C₆₀ fullerenes, also known as buckminsterfullerenes or ‘buckyballs,’ are of particular interest. These are spherical closed-form fullerenes consisting of 60 carbon atoms. However, other-form and other-sized (e.g. C₂₀, C₄₀, etc.) fullerenes also may find effect in the disclosed methods. C₆₀ fullerenes are preferred in part because they are well known and well-characterized, and are relatively more available than other forms. Moreover, C₆₀ fullerenes also are highly coherent, spherical molecules that exhibit high vibrational coherence. This makes them good photoacoustic agents because their near-perfect symmetry results in their primary mode of energy-delivery upon irradiation being acoustic (vibratory), and not necessarily thermal. Other, less-symmetric fullerenes also may be used, wherein the energy-delivery mechanism of the molecules upon irradiation would be expected to become increasingly thermal (relative to acoustic) with decreasing molecular symmetry. As a result, lower-symmetry fullerenes may impart a greater proportion of thermal energy to adjacent or contacting kidney stones upon irradiation, which could increase the proportion of thermal-energy delivery (as opposed to acoustic energy-delivery) to those stones to produce smaller-sized fragments.

Currently preferred photonic nanoparticles used in the present methods will be constituted by molecules that possess a high degree of 3-dimensional symmetry. Preferably, the nanoparticle molecules will be symmetric with respect to one of the following point groups: Cₛ, Cₙᵥ, Dₙₕ, T_{d}, Oₕ, Iₕ or Kₕ. Such 3-dimensional symmetry will result predominantly in an acoustic energy-delivery mechanism upon IR-irradiation. Among such particles, C₆₀ fullerenes discussed above are preferred, in part because they are readily available. Other highly symmetric fullerenes having different numbers of carbon atoms also may be used, wherein it is relatively simple to design symmetric molecules composed of carbon-atom rings. In addition, other 3-dimensionally symmetric photonic nanoparticles (e.g. gold nanoshells (AuNS—which have Kn symmetry) that are not fullerenes also may be useful.

Alternatively, photonic nanoparticles having lower symmetry or a high aspect ratio also may be used. Lower-symmetry photonic nanoparticles will exhibit a greater degree of thermal (as opposed to mechanical/acoustic) energy delivery (essentially in proportion to their degree of asymmetry) upon near-IR irradiation. Examples include gold nanorods (AuNR), graphene, and carbon nanotubes (CNT). In any event, the selected nanoparticles should be excitable to deliver energy in the form of work, heat or both to an adjacent or adhered kidney stone upon near-IR irradiation within a known IR biological transparency window, such as those mentioned above. This will assure that the near-IR laser used to activate the selected nanoparticles will conform to wavelengths to which the surrounding human tissue will be substantially transparent; so that those tissues will not absorb significant energy from irradiation during treatment. Ideally, the nanoparticles also will be selected so that laser power at the selected tissue-transparent wavelength (e.g. 785 nm) of <5 W (most preferably 0.1 to 2 W, or 0.5 W to 1 W, when laser energy is delivered via a retrograde approach) will be effective to initiate nanoparticle-energy delivery (e.g. vibration via phonon generation).

As discussed below, the photonic nanoparticles (e.g. fullerenes, such as C₆₀ fullerenes) generally will be delivered to a treatment site where they will be placed in the vicinity of, preferably into contact with, a kidney stone requiring treatment. To be most effective (i.e. to deliver a maximum proportion of their generated energy to the stone in order to fragment it), the nanoparticles should be in contact with the stone. As used herein a photonic nanoparticle is considered to ‘contact’ or be ‘in contact with’ a kidney stone if it is either in direct physical contact with the stone, or suspended in a suspension medium that itself is in physical contact with the stone, such as when the stone has been coated with the suspension comprising the nanoparticles in the suspension medium. This can be achieved by adapting the delivery vehicle (e.g. the suspension medium) so that it will adhere to and coat the stone. Nanoparticle-to-stone contact also can be achieved or augmented by adapting the nanoparticles themselves so that they will interact with the stone in an adhesive manner.

For example, the nanoparticles may be functionalized with ligands known or designed to interact with common kidney-stone compositions, so that the resulting interactions will produce adhesion therewith. In one example, hydroxyl groups can be decorated over the surface of fullerene molecules to yield polyhydroxy fullerene or ‘PHF.’ Most commonly, PHF is available and supplied as the C₆₀-mer of fullerene (i.e. buckminsterfullerene), and oftentimes ‘PHF’ generally is understood to refer to the OH-functionalized C₆₀-mer absent contrary indication. However, PHF particles useful in the instant methods need not necessarily be the C₆₀-mer.

Pristine fullerenes are strongly hydrophobic, non-polar molecules that would exhibit minimal native interaction with most kidney stones, which usually are mineral (and thus highly polar) in nature. But fullerenes functionalized with oxygen-containing functional groups (e.g. PHF) possess polar sites decorated over their surface, which can interact with polar moieties at the surface of a mineral-based stone to yield adhesion. For example, calcium oxalate- and calcium phosphate-based stones represent at least 80% of the kidney stones requiring treatment. These are polar salts. PHF nanoparticles have been shown to strongly adhere to such stone compositions via electrostatic interactions.

In example embodiments, functionalized fullerene particles useful in the disclosed methods can comprise a compound according to the formula C₂ₙ(OH)ₜ(SH)ᵤ(NH₂)ᵥ(COOH)_{w}(COOM)ₓO_{y}M_{z}, wherein: M is an alkali metal, alkaline earth metal, transition metal, post-transition metal, lanthanide, or actinide; n is a number ranging from 10 to 270, t is number ranging from 0 to 60, u is a number ranging from 0 to 60, v is a number ranging from 0 to 60, w is a number ranging from 0 to 60, x is a number ranging from 0 to 60, y is a number ranging from 0 to 30, and z is a number ranging from 0 to 30. In one embodiment, the fullerene composition is selected from the group consisting of: C₆₀ (OH)₉O₇Na₆; C₆₀ (OH)₁₁O₈Na₅; C₆₀ (OH)₁₁O₁₂Na₈; C₆₀ (OH)₁₁O₂₀Na₁₀K₆; C₆₀ (OH)₆O₄Na₄; C₆₀ (OH)₂₀O₈Na₄; C₆₀ (OH)₁₀O₁₃Na₆; C₆₀ (OH)₄O₁₄Na₁₇; C₆₀ (OH)₁₃O₄Na₃; C₆₀ (OH)₁₀; C₆₀ (OH)₂₂₋₂₄; C₆₀(OH)₃₆; C₆₀(OH)₄₄; C₆₀O₁₃Na₁₄; Gd@C₈₂(OH)₁₅O₁₂Na₅; and Gd₃N@C₈₀(OH)₁₃O₉Na₆. In some embodiments, the functionalized fullerene is a PHF according to the formula C₂ₙ(OH)ₜO_{y}M_{z}, which corresponds to the broader formula disclosed earlier in this paragraph wherein u. v, w, and x are 0. In other embodiments, a polyhydroxy fullerene is a compound according to formula C₂ₙ(OH)ₜ, which corresponds to a functionalized fullerene wherein t is 1 to 60, and u, v, w, x, y, and z are 0. In further embodiments, a polyhydroxy fullerene is a compound according to the formula C₂ₙ(OH)ₜ(COOH)_{w}O_{y}M_{z}, which corresponds to a functionalized fullerene as in the first formula given in this paragraph wherein u, v and x are 0. The specific variants discussed in this paragraph are examples of functionalized fullerenes encompassed by the formula also disclosed herein, and are not meant to limit the scope of the claims to these specific examples.

Fullerene molecules also can be functionalized with other ligands, which may be selected and tuned to bind to the composition (if known) of a particular target stone based on its chemical makeup. For example, bisphosphonate ligands may be decorated onto fullerenes, including PHF. Bisphosphonate ligands target and bind with calcium, and thus can serve as strong adhesion promoters with the most common, calcium-based kidney stones. Still other ligands (e.g., containing polyanionic moieties, such as carboxylate or sulfonate groups in addition of phosphate groups) may be useful and could be selected by the skilled artisan for a given application, where the composition (or likely composition) of the kidney stone is known or can be deduced from clinical or diagnostic examination.

The foregoing discussion was given primarily in relation to C₆₀ (Buckminster)fullerenes. However, other photonic nanoparticles useful in the disclosed methods, including other fullerenes and other non-fullerene nanoparticles (several examples of which are provided above), also can be similarly decorated or functionalized to impart the described features or functionality to the particles when used.

### Delivery Methodologies

In order to deliver the nanoparticles, a suspension of the particles can be prepared and then introduced into the vicinity of the stone, within the kidney or ureter. A first delivery modality is transcutaneous, where a suspension of the photonic nanoparticles is prepared and filled into a syringe, which is used to inject them through the skin and into the kidney in the vicinity of the kidney stone. This procedure may be indicated, for example, when the stone is in a location within the kidney that would be readily accessible via needle puncture along a transcutaneous path without having to penetrate nearby (or critical) biologic structures or organs. If indicated, fluoroscopy, ultrasound or other suitable imaging techniques may be utilized to provide needle guidance for transcutaneous delivery of the photonic nanoparticles, similarly as in other needle-guidance procedures.

Once the needle has been properly positioned adjacent to the kidney stone, the syringe is actuated to deliver the particle suspension, and then the needle is withdrawn. As noted above, the particles ideally will be in contact with the stone in order to yield the greatest effectiveness to fragment the stone upon activation of the particles.

A second, and likely preferred, delivery modality is retrograde delivery of the nanoparticles via the urethra, bladder and ureter. Retrograde delivery of other diagnostic or therapeutic agents and instruments for treating kidney stones (e.g. via conventional ureteroscopy), as well as other conditions within the kidney, are well-known and well-practiced interventions for clinicians. These techniques are routinely performed in the operating room, but some can also be done in the clinician's office with local anesthesia, depending on the patient and the specific scenario. The need for image guidance, such as with ultrasound or fluoroscopy, will depend on the position of the kidney stone as well as surgeon preference. For example, if the stone is in the kidney, and the surgeon feels comfortable using anatomic landmarks to target the IR energy, then no imaging will be needed. Alternatively, if the stone is in the ureter, then it may require more specific targeting with ultrasound or fluoroscopy based on the particular surgeon's practice. Thus using the disclosed methods, there will be situations where imaging may no longer be required compared to conventional ureteroscopy, thus potentially reducing the need for ionizing radiation. Moreover, retrograde delivery will be available with less regard to the position of the stone within the kidney (relative to transcutaneous delivery) and does not risk damaging biologic structures external to the kidney and the urinary system. It is for these reasons that retrograde delivery is likely to be preferred to transcutaneous delivery in many applications.

For retrograde delivery a small catheter can be placed into the ureter, via the urethra and bladder, and guided optically via a conventional cystoscope. The catheter is advanced through the ureter so that its proximal end approaches the stone; it need not contact the stone. If the stone is in the ureter, then the proximal end may be advanced until it reaches the vicinity of the stone. If the stone is in the kidney beyond the ureter, the proximal end of the catheter may be advanced until it emerges within the renal pelvis of the kidney, and optionally further to position it in the vicinity of the stone inside the kidney (e.g. under optical or image guidance). Once properly positioned, the nanoparticulate suspension can be delivered into the vicinity of the stone via injection through the catheter, so that the suspended particles will adhere to the stone.

If the stone is in the ureter, a relatively small volume of nanoparticle suspension (e.g. <5 cc, or <1 cc) can be suitable to coat the stone, located in the vicinity of the catheter's proximal end. If the stone(s) is/are inside the kidney, either in the renal pelvis or in one (or more) of the adjacent calyces, then it can be effective to inject sufficient nanoparticle suspension (e.g. 10 cc to 20 cc) to fill that volume. In this manner, functionalized nanoparticles (or medium) designed to adhere to the stone-surface composition will come into contact therewith and adhere thereto wherever within the kidney it/they may be located. Once the suspension medium (or nanoparticles thereof) has (have) had sufficient time to achieve stone-adherence (e.g. 1, 5, 10, or 15 minutes), excess suspension medium can be flushed from the kidney prior to laser irradiation. This can be either through natural urine production and excretion from the kidney, which may take from 1 to 30 minutes. Alternatively, the operator may manually flush the kidney with saline or other appropriate reagent to remove excess suspension medium and nanoparticles, using the same catheter that was used to deliver the nanoparticle suspension.

If desired, a biocompatible gel (devoid of photonic nanoparticles) can be delivered retrograde into the kidney after the functionalized nanoparticles have been delivered and adhered to present kidney stones. Once delivered, the gel material gelatinizes to form a semi-solid gel material that substantially fills the void volume (e.g. renal pelvis and calyces) within the kidney, effectively encapsulating the treated kidney stones so that the previously adhered photonic nanoparticles will remain in-place throughout treatment. This may be desired, for example, to ensure the functionalized particles do not become detached or dislodged from the adhered stones, e.g. through chemical degradation of the functionalizing species or other mechanisms. Such a gel also will ensure that kidney stones remain in-place (e.g. so that they do not shift during treatment). One such gel that has been developed for use within the kidney is Jelmyto®, a reverse thermal hydrogel available from UroGen Pharma Ltd. This gel can be delivered to permeate the kidney voids, where it will solidify and remain in-place for 4-6 hours prior to re-liquifying and being eluted through urine excretion. During that time laser-irradiation treatment can be carried out to excite the photonic nanoparticles adhered to present kidney stones, in order to fragment the stones. Once the gel liquefies, the stone fragments can be excreted together with the gel residue in urine.

Once the nanoparticles have been delivered (and any flushing performed using the catheter to remove excess nanoparticles/suspension from the kidney, and/or delivery of any desired gel encapsulation), the catheter and cystoscope may be removed. Alternatively, the catheter may remain in-place to guide a second-round delivery of nanoparticles if the first-round treatment (discussed below) does not eradicate the stone via sufficient fragmentation.

A third delivery modality is intravenous, wherein a suspension of photonic nanoparticles can be prepared and injected intravenously into the patient. For intravenous administration, preferably the nanoparticles have a mean particle size not greater than 5 nm. Particles of this size can be filtered from the blood by the kidney, and delivered into the calyces and renal pelvis via the normal biologic urine-metabolism of the patient. For example, one of the inventors has found (via MRI visualization) that this process can occur for Gd@C₈₂ PHF injected intravenously into mice within thirty minutes. The IV-administration suspension medium can be any suitable medium for IV-injection; such as sterile water or saline. Once the photonic nanoparticles have been filtered by the kidney and delivered to the calyces, renal pelvis and ureter, they can adhere to any kidney stone present therein. When delivered via the intravenous modality, the nanoparticles preferably are functionalized in order that they will adhere to the (known or presumed) surface composition of the stone(s) requiring treatment. Residual nanoparticles that do not ultimately adhere to present kidney stones typically would be flushed by the kidney through urine production within five to thirty minutes.

Regardless of the delivery modality, when the nanoparticles have been functionalized to promote adhesion to the stone, no special adaptation of the suspension medium (so that the medium itself will adhere to the stone) may be called for. Rather, any conventional and biocompatible (preferably non-immunogenic) medium may be used regardless of its adhesive properties (or lack thereof), including water, other aqueous solutions, etc.

Alternatively, and particularly when the nanoparticles do not possess features that will promote adhesion to the kidney stone, when nanoparticle delivery is to be percutaneous or retrograde the suspension medium can be selected or tuned to provide such adhesion. In particular, biocompatible gels can be utilized as the suspension medium such that upon delivery in the vicinity of the stone, the gel will coat and adhere to the stone, thereby presenting a nanoparticle coating to the stone. Such coating can be provided as a function of the inherent viscosity and/or adhesive characteristics of the gel selected. In this manner the nanoparticles are held in contact with the stone via the gel, which acts as a glue. Suitable gels can include all nature of biocompatible (preferably non-immunogenic) gels known in the art, including but not limited to polymeric gels such as PLA, PGA, etc., as well as hydrogels such as (or based on) hyaluronic acid, alginates, chitosan, etc. High-viscosity/adhesive gels generally will be inappropriate suspension media for intravenous delivery, because such gels may obstruct the patient's circulatory system. Moreover, the biological process of filtering blood to produce urine will separate the photonic nanoparticles from the circulating fluid/medium, and alter the character of the medium prior to delivery into the renal pelvis through urine production. This is why functionalized-particle adhesion is preferred for promoting adhesion of the photonic nanoparticles to the kidney stone(s) when delivered via IV-administration.

Whether via functionalized ligands, adhesive gel, or both, upon delivery of the photonic nanoparticles in the vicinity of the stone, the nanoparticles are caused to adhere to and contact the stone where they will be effective to fragment the stone upon irradiation of the nanoparticles in the irradiation step discussed below.

### Irradiation of Photonic Nanoparticles for Treatment of Kidney Stones

With the photonic nanoparticles in contact with the kidney stone, those nanoparticles are irradiated to facilitate activation and release of acoustic and/or thermal energy. Because they are in contact with the kidney stone, the resulting energy is delivered to the stone, which is effective to fragment it.

Delivery of the irradiation energy to activate the nanoparticles can be from an external emitter that supplies that energy for delivery transcutaneously, or the energy can be delivered from the external emitter retrograde to the vicinity of the stone, similar to conventional holmium and thulium fiber contact lasers presently used to treat stones via ureteroscopy. A key difference, in addition to the fact that contact between the laser and the stone is not required, is that because, at least in the case of the preferred fullerene nanoparticles, those particles will release vibrational energy in response to wide-spectrum irradiation, IR wavelengths that exhibit low or minimal absorption by water and biological tissues can be used. For example, laser energy delivered at a wavelength of 785 nm is effective to activate fullerene nanoparticles, such as C₆₀ PHF. Laser energy at this wavelength also can be effective to activate fullerenes at much lower power than conventional thulium fiber and holmium contact lasers, e.g. 0.1 W to 5 W, 0.5 W to 5 W and particularly 0.5 W to 1 W, compared to 20 W to 120 W power settings most commonly used for conventional contact lasers required to fragment a kidney stone directly. Other photonic nanoparticles also can be selected that will meet the aforementioned or similar criteria in order that high-powered laser energy is not required, and that such energy can be delivered within an aforementioned tissue-transparency window.

The comparably lower water-absorptivity (holmium and thulium fiber lasers operate at ˜2100 and 1940 nm wavelengths, which are well-absorbed by water), and much lower power of the preferred (e.g. 785 nm) infrared laser energy compared to conventional contact lasers result in lower risk of adjacent tissue- or organ damage from heat mediated injury to cells or direct damage from the laser itself. In particular, because water molecules will not efficiently absorb low-infrared radiation supplied by the preferred laser emitters, heat generation within surrounding tissues from the laser is minimized (i.e. is substantially negligible during the procedure) compared to conventional contact lasers, which strongly excite water molecules and can generate significant heat. Moreover, because using the disclosed methods the energy is delivered to irradiate nanoparticles contacting the stone surface, no part of the laser emitter need contact the stone directly to be effective (unlike the thulium fiber and holmium lasers, which require contact with the stone). Rather, it need only deliver the infrared radiation energy to the photonic nanoparticles (e.g. PHF), which will generate vibrational and/or thermal energy targeted to the contacted kidney stone upon being irradiated, thereby fragmenting the kidney stone.

When delivered via a retrograde catheter-form emitter, laser energy at 785 nm may be sufficient to activate photonic nanoparticles at very low power; e.g. 0.1 to 2 W, or 0.5 to 1 W, from greater than 3 mm or 5 mm away from the stone, up to several centimeters away; e.g. a range of 1-10 cm, 1-7 cm, or 1-5 cm from the stone. When delivered transcutaneously from outside the body, the power at the emitter may need to be adjusted to be higher, in order that sufficient energy penetrates to the depth of the stone within the kidney, beneath the patient's skin and through intervening tissue; e.g. up to 5 W from a range to the stone of 5-20 cm, or 5-15 cm. Even so, this is still comparably low power compared to conventional contact laser emitters.

Another benefit of using such a low-powered laser as herein described is that the entire laser emitter may be a portable, handheld unit, even battery-powered, without the need for special cooling systems such as the standard holmium laser used presently. Rather, the laser emitter can be simply fan-cooled (e.g. via an onboard fan) without requiring a liquid-cooling circuit. Such a small, low-power system also will produce less noise compared to conventional contact-laser emitters; e.g. <about 30 dB, compared to ˜60-70 dB for conventional emitters, materially reducing the noise level experienced by both the operator and the patient during lithotripsy. This reduced noise can protect the operator's hearing during successive procedures, as well as improve communication between the operating team.

Ideally the stone undergoing treatment will be completely covered with photonic nanoparticles (e.g. the stone will be enveloped within a suspension medium carrying the nanoparticles, which can be an adhesive gel), so that upon irradiation with IR energy the photonic nanoparticles generate vibrations that are compounded to yield a cascading effect that implodes the kidney stone into tiny fragments that will be essentially imperceptible upon spontaneous passage. Complete coating of the kidney stone within the kidney may be achieved by simply positioning the suspension-delivery catheter in the vicinity of the stone, and delivering sufficient suspension in that vicinity to coat the stone. This may be achieved without the standard instrumentation required during ureteroscopy, such as a ureteral access sheath or even a ureteroscope. Such a sheath or instrument manipulations may irritate the ureter and yield post-procedure swelling and inflammation. Accordingly, it also may be possible to omit the highly-bothersome ureteral stent often used to ensure post-procedure patency, which otherwise may be restricted due to swelling from ureteral inflammation.

As noted above, the coating-and-irradiation procedure can be carried out in a first round and then the result observed via imaging or visually via an emplaced ureteroscope, depending on the technique being used. If fragmentation was complete following the first round, the procedure will conclude and the patient will be discharged. If, however, fragmentation is incomplete then the treatment may be repeated for a second round, or even successive rounds, if necessary.

As discussed above, the disclosed methods possess several advantages. For example, the near-IR laser used to irradiate the nanoparticles in the vicinity of (preferably in contact with) the kidney stone, need not itself physically contact the stone for effective treatment. Rather, the laser emitter can be operated in a non-contact mode, such that it is spaced more than 3 mm away, more than 5 mm away, or up to several centimeters away from the stone, from which it can deliver near-IR energy to the nanoparticles in its vicinity or in contact therewith. Accordingly, direct visualization of the stone may not even be needed, unlike for both ESWL and conventional laser lithotripsy. Also, the use of a low-intensity, IR laser (e.g. <5 W or <1 W via transcutaneous or retrograde energy delivery, respectively), results in a lower power system that can minimize heat generation from the laser during treatment, which will reduce the risk of damage to healthy tissues from the laser emitter in the treatment vicinity. Because the nanoparticles deliver their energy directly to stones in their vicinity or in contact therewith, and because laser-contact of the stone itself is not required, the disclosed methods have the potential to greatly improve efficiency and reduce the duration of treatment for each lithotripsy procedure. Moreover, the need for fluoroscopy (e.g. typically used for ESWL and conventional ureteroscopy) can be minimized or even eliminated, because fluoroscopic guidance will not be required to directly visualize the stone or to pass numerous instruments into the kidney. Instead, non-contact remote operation (e.g. greater than 3 mm, up to several centimeters from the stone) can be effective to activate the photonic nanoparticles in the vicinity of or in contact with the kidney stone.

### EXAMPLES

### Example 1

A proof-of-concept in vitro experiment was conducted on kidney stones previously removed from patients to evaluate fragmentation/comminution performance on the stones using photonic lithotripsy methods via irradiation of photonic nanoparticles. Specifically, human kidney stones composed of calcium oxalate monohydrate (COM), calcium oxalate dihydrate (COD), calcium phosphate (CaP), uric acid (UA) or their mixtures were obtained from Cleveland Clinic Pathology Labs. The stones were treated (or not, as indicated below) with C₆₀ PHF from e.g., Suzhou Dade Carbon Nanotechnology and then irradiated by a near-infrared laser (785 nm; B&W Tek, Newark, DE) for 5 minutes at a distance of 10 mm. To apply the fullerene particles, 10 μl of a suspension of C₆₀ PHF (10 mg/mL) particles suspended in either water or urine simulant was pipetted directly onto each kidney stone. Fragmentation was measured as a function of the following three parameters: laser dose (0.5-4 W); nanoparticle treatment time (0-24 hr); laser treatment time (0-10 min); and stone size (3-10 mm).

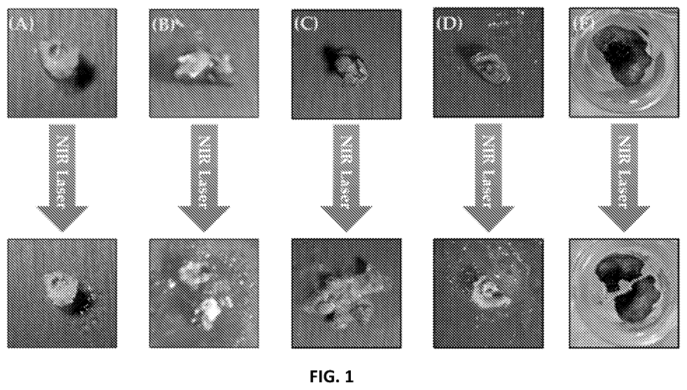

Results are shown in FIG. 1, which reflect the experimental treatment of kidney stones as described above under the following conditions:
Stone (A) was composed of 50% calcium phosphate (CaP), 40% calcium oxalate monohydrate (COM) and 10% uric acid (UA), all by weight. It was untreated, meaning it had no PHF nanoparticles in contact therewith prior to laser irradiation.
Stone (B) had the same composition as Stone (A), but was treated with the C₆₀ PHF nanoparticles via pipetting as noted above, and allowed to soak in the PHF suspension for thirty minutes prior to laser irradiation.
Stone (C) was composed of 50% calcium oxalate dihydrate (COD) and 50% calcium phosphate (CaP), both by weight. It was treated with the C₆₀ PHF nanoparticles via pipetting as noted above, and then allowed to soak in the PHF suspension for thirty minutes prior to laser irradiation.
Stone (D) was composed of 100% uric acid (UA) and was treated with the C₆₀ PHF nanoparticles via pipetting as noted above, and then allowed to soak in the PHF suspension for thirty minutes prior to laser irradiation.
Stone (E) was the same as Stone (D), which did not materially fragment following the first treatment and laser-irradiation thereof. Accordingly, additional C₆₀ PHF nanoparticles were delivered via pipetting (100-200 μl at 10 mg/ml) the nanoparticle suspension, and the stone was allowed to soak in that suspension for an additional 24 hours prior to laser-irradiation a second time.

All of Stones (A) through (E) were irradiated using an infrared laser emitting at a power of 0.5 W and a wavelength of 785 nm, from a distance of 10 mm for a duration of five minutes. In addition, Stone (A) (the untreated stone) was laser-irradiated a second time at a power of 1 W at 785 nm from a distance of 10 mm for a duration of five minutes.

Comparing Stones (A) and (B), which were of the same CaP/COM/UA composition, PHF pre-treatment of the stone produced substantial fragmentation in Stone (B); whereas the untreated stone (Stone (A)) remained substantially intact post-irradiation.

Comparing Stones (D) and (E), which were the same stone composed of 100% UA, as noted above the initial treatment and irradiation (promptly after delivering the photonic PHF nanoparticles) failed to fragment the stone. However, following re-treatment and equilibration with the PHF suspension for a 24-hour period, the 100%-UA stone was significantly fragmented following a second round of laser-irradiation.

Finally, Stone (C) exhibited substantial fragmentation following irradiation.

Notably, although post-irradiation stones (B), (E), and to a lesser extent (C), all appear to comprise relatively large fragments of the original stone, in fact those large fragments all were substantially friable, composed of smaller particulates that together yielded the observed larger particle aggregates. But those larger aggregates would be immediately and readily separable into the constituent smaller particles with minimal external force, e.g. hydrodynamic forces as would be present in a fluidic suspension or stream—such as the prevailing environment within a patient's urinary tract, including the kidneys.

These results demonstrate the efficacy of the disclosed methods using photonic nanoparticles irradiated with non-ionizing electromagnetic radiation (e.g. IR laser light) from a distance, to fragment kidney stones of a variety of compositions derived from the most common compounds (and mixtures thereof) that make up human kidney stones.

### Example 2

Additional in vitro studies were carried out on six more kidney stones, all having similar compositions and obtained from Cleveland Clinic Pathology Labs, namely 90% calcium oxalate (mixture of mono- and di-hydrate), 10% uric acid. These stones all were of comparable size (˜4 mm), and were treated similarly with PHF as follows. The stones were soaked in 0.2 mL of a 10 mg/mL C₆₀ PHF suspension (in water as the suspension medium) for 24 hours. Then the stones were placed on a Petri dish and 10 uL of the PHF suspension was pipetted on top of each stone. Each stone then was irradiated with laser light for a duration of three minutes. The laser irradiation was at 1320 nm wavelength at 4 W.

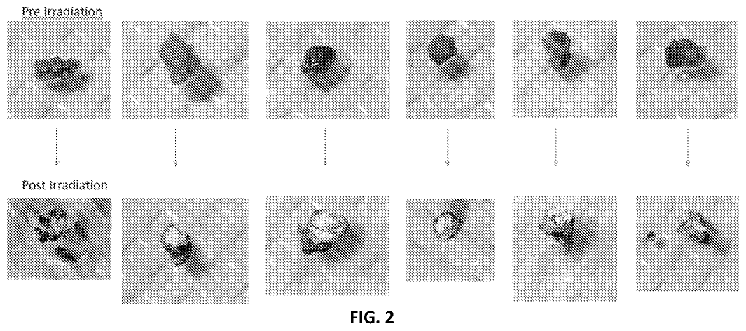

The pre- and post-irradiation results are shown for the six stones in FIG. 2. Fragmentation in all cases was observed within three minutes of the start of irradiation. Following irradiation, each stone was markedly fragmented, each with a white mineral appearance having been introduced at the site of laser irradiation. See FIG. 2, where the post-irradiation images of stones 1 through 6 reflect a white mineral layer at the irradiation site. Thermal imaging of other stones that were similarly treated (and which also yielded a similar white residue as in this example) indicated that such kidney stones coated in PHF can reach elevated temperatures of at least 660° C. during laser irradiation at the irradiation sites. Moreover, comparative pre- and post-irradiation FTIR analyses of several of the stones in this example confirmed that the observed white mineral on those stones was calcium carbonate, believed to have been formed via thermal decomposition of calcium oxalate in the original stone. The aforementioned thermal-imaging studies as well as the formation of calcium carbonate suggest that even when highly symmetrical C₆₀ PHF is used as the photonic nanoparticle, whose primary energy-delivery mechanism on irradiation is acoustic and not thermal, thermal effects nonetheless may play a role in kidney-stone fragmentation. The precise mechanism of this is not known.

### Example 3

Still further in vitro studies were carried out on six more kidney stones, all having similar compositions and obtained from Cleveland Clinic Pathology Labs, namely 50% calcium oxalate monohydrate (COM) and 50% uric acid (UA). These stones again were of comparable size, and were treated similarly with PHF as follows. The stones were treated similarly as in Example 2, including laser irradiation at 1320 nm wavelength at 4 W for three minutes.

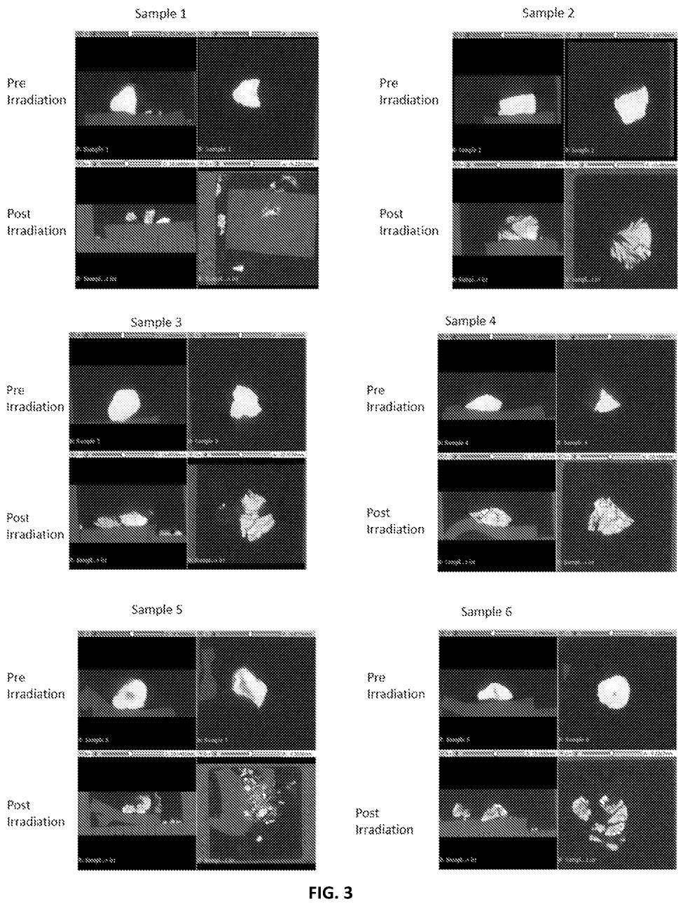

As in the prior example, the post-irradiation stones appeared to comprise relatively large stone fragments. But in fact those fragments were shown to be aggregates of smaller stone particles that had yet to be physically separated due to a lack of external separation force. FIG. 3, shows pre- and post-irradiation micro-CT images of the stones 1 through 6 in this example. The samples were scanned using the following parameters; Tube voltage: 80 kV, tube current: 490 μA, 360°, 360° scan and effective resolution of 20 m. The post-irradiation images clearly show that cracks propagated throughout the interior of each ostensive large stone fragment. Those fragments thus themselves were fragmented into smaller particulates that will separate upon application of a minimal external force. The resulting smaller particulates will more easily pass through small-diameter passages (such as a patient's urinary tract) than the original stone (or the ostensive larger stone fragments) prior to treatment as disclosed.

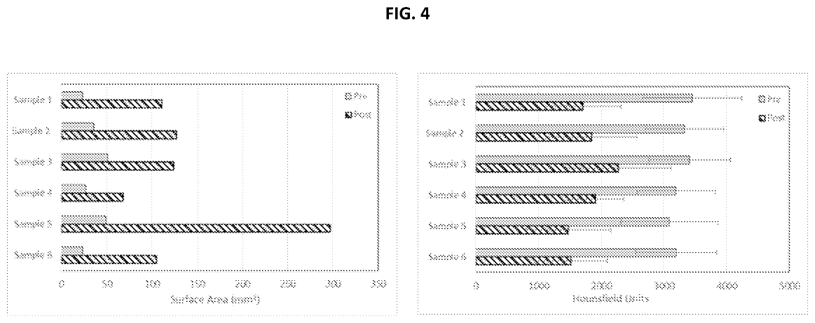

These results are further confirmed via Hounsfield-unit studies conducted on stones 1 through 6 in this example, the results of which are reported at FIG. 4. As seen there, each stone treated in this example exhibited a roughly 30% to 50% decrease in Hounsfield units, reflecting materially reduced density in each stone post-treatment. Concordantly, those stones exhibited between a 2-fold and 6-fold increase in specific surface area, consistent with propagation of internal cracks and the formation of sub-particulates within each original stone, which contributed to the larger overall specific surface area.

### Example 4

Still further in vitro studies were carried out on additional kidney stones to demonstrate efficacy of the disclosed methods using less symmetric photonic nanoparticles than C₆₀ fullerenes. In this example, five further stones obtained from Cleveland Clinic Pathology Labs all were of comparable size. The particular stone compositions, and of the nanoparticle suspensions used to treat the respective stones, are given below:
a) C₆₀ PHF suspended in water (concentration of 10 mg/mL); Stone type (70% calcium oxalate monohydrate (COM), 20% calcium oxalate dihydrate (COD), 10% uric acid (UA)
b) Graphene (GOx) oxide suspended in water (concentration of 0.015 mg/mL); Stone type (70% uric acid (UA), 30% calcium oxalate monohydrate (COM)
c) Multi-walled carbon nanotubes (MWNT) suspended in water (concentration of 0.015 mg/mL); Stone type (70% uric acid (UA), 30% calcium oxalate monohydrate (COM)
d) Gold nanorods (AuNR) suspended in water (concentration of 0.04 mg/mL); Stone type (70% calcium oxalate mononydrate (COM), 20% calcium oxalate dihydrate (COD), 10% uric acid (UA)
e) Gold nanoshells (AuNS) suspended in water (concentration of 0.015 mg/mL); Stone type (60% calcium oxalate monohydrate (COM), 30% calcium oxalate dihydrate (COD), 10% uric acid (UA)

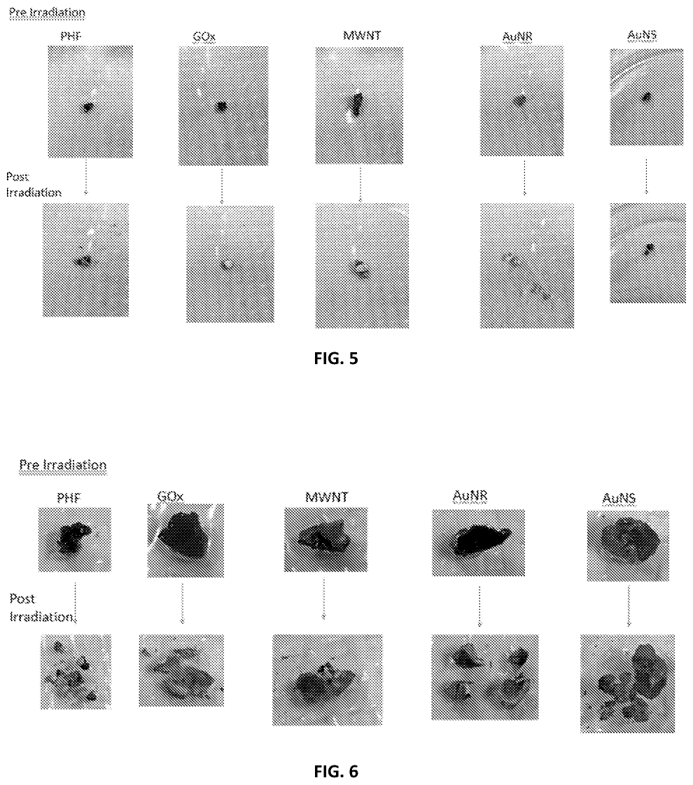

These stones again were of comparable size, and were treated similarly as in Example 2, except that here the above-noted nanoparticle suspension were used and that laser irradiation was at 1320 nm wavelength at 3 W (instead of 4 W) for three minutes. The results are shown in FIG. 5, where each of the stones can be seen to have fragmented following laser irradiation for three minutes as described.

### Example 5

Still further in vitro studies were carried out on additional kidney stones to demonstrate efficacy of the disclosed methods using the same photonic nanoparticles as-used in Example 4, but at a different wavelength of laser light used for irradiating the stones. The particular stone compositions, and of the nanoparticle suspensions used to treat the respective stones in this example, are given below:
f) C₆₀ PHF suspended in water (concentration of 10 mg/mL); Stone type (70% calcium oxalate monohydrate (COM), 20% calcium oxalate dihydrate (COD), 10% uric acid (UA)
g) Graphene (GOx) oxide suspended in water (concentration of 0.25 mg/mL); Stone type (50% calcium oxalate dihydrate (COD), 50% uric acid (UA)
h) Multi-walled carbon nanotubes (MWNT) suspended in water (concentration of 0.03 mg/mL); Stone type (70% calcium oxalate monohydrate (COM), 20% calcium oxalate dihydrate (COD), 10% uric acid (UA)
i) Gold nanorods (AuNR) suspended in water (concentration of 0.015 mg/m); Stone type (70% calcium oxalate monohydrate (COM), 20% calcium oxalate dihydrate (COD), 10% uric acid (UA)
j) Gold nanoshells (AuNS) suspended in water (concentration of 0.06 mg/mL); Stone type (50% calcium oxalate dihydrate (COD), 50% uric acid (UA)

The stones in this Example 5 were irradiated using laser light at a wavelength of 785 nm at 2 W. Otherwise, the laser-treatment protocol was the same as in Example 4. The results are shown in FIG. 6, where again each of the stones can be seen to have fragmented following laser irradiation for three minutes as described.

Although the invention has been described with respect to selected embodiments, it is to be understood that the invention is not limited thereby, but rather is intended to encompass all embodiments and features falling within the spirit and the scope of the invention as set forth in the appended claims.

1. A combination comprising a kidney stone with a photonic nanoparticle at a surface of the kidney stone, said photonic nanoparticle beinq a polyhydroxy fullerene (PHF).

2. The combination of claim 1, said photonic nanoparticle being in contact with the kidney stone at its surface.

3. (canceled)

4. (canceled)

5. (canceled)

6. The combination claim 1, said photonic nanoparticle being suspended in a biocompatible gel to yield a nanoparticle suspension, the nanoparticle suspension at least partially coating the kidney stone.

7. The combination of claim 1, said nanoparticle being comprised of molecules that are symmetric with respect to one of the following point groups: Cₛ, Cₙᵥ, Dₙₕ, T_{d}, Oₕ, Iₕ or Kₕ.

8. The combination of claim 1, said photonic nanoparticle being a functionalized fullerene particle of the formula C₂ₙ(OH)ₜ(SH)ᵤ(NH₂)ᵥ(COOH)_{w}(COOM)ₓO_{y}M_{z}, wherein: M is an alkali metal, alkaline earth metal, transition metal, post-transition metal, lanthanide, or actinide; n is a number ranging from 10 to 270, t is number ranging from 0 to 60, u is a number ranging from 0 to 60, v is a number ranging from 0 to 60, w is a number ranging from 0 to 60, x is a number ranging from 0 to 60, y is a number ranging from 0 to 30, and z is a number ranging from 0 to 30.

9. The combination of claim 1, said photonic nanoparticles being functionalized with a ligand that interacts with a predetermined composition of said kidney stone, thereby producing adhesion between the nanoparticles and the kidney stone.

10. (canceled)

11. The combination of claim 1, said nanoparticle being excitable via irradiation with non-ionizing radiation, and thereby effective to deliver energy to the kidney stone to fragment said stone.

12. The combination of claim 11, said non-ionizing radiation being infrared radiation at not more than 5 W.

13. The combination of claim 11, said non-ionizing radiation being infrared laser radiation at not more than 5 W.

14. The combination of claim 11, said non-ionizing radiation being infrared radiation within one of the following wavelength ranges: 700-950 nm, 1000-1350 nm, 1600-1870 nm, 2100-2300 nm.

15. A method of lithotripsy comprising delivering photonic nanoparticles in a vicinity of a kidney stone within a patient, and thereafter irradiating said nanoparticles with non-ionizing electromagnetic radiation to fragment the kidney stone.

16. The method of claim 15, wherein said nanoparticles yield acoustic energy in response to being irradiated, and thereby perform work on the kidney stone to fragment the kidney stone.

17. The method of claim 15, wherein in response to being irradiated said nanoparticles yield at least one of acoustic energy or thermal energy delivered to the kidney stone.

18. The method of claim 15, said kidney stone being within a kidney of the patient, said photonic nanoparticles being delivered retrograde, in the vicinity of the kidney stone within the kidney.

19. The method of claim 15, said kidney stone being within a kidney of the patient, said photonic nanoparticles being delivered intravenously, in the vicinity of the kidney stone within the kidney.

20. The method of claim 15, said kidney stone being within a kidney of the patient, said photonic nanoparticles being delivered percutaneously, in the vicinity of the kidney stone within the kidney.

21. The method of claim 15, said kidney stone being within a ureter of the patient.

22. The method of claim 15, said photonic nanoparticles being in contact with said kidney stone prior to activation thereof.

23. The method of claim 15, said irradiation of said nanoparticles being delivered retrograde via a laser operating at an infrared wavelength and at not more than 5 W.

24. The method of claim 15, said irradiation of said nanoparticles being delivered retrograde via a laser operating at an infrared wavelength and at not more than 1 W.

25. The method of claim 15, said irradiation of said nanoparticles being delivered transcutaneously via a laser operating at an infrared wavelength and at not more than 5 W.

26. The method of claim 15, said irradiation of said nanoparticles being delivered via infrared radiation at a wavelength at which human soft tissues are substantially transparent thereto.

27. The method of claim 15, said irradiation of said nanoparticles being delivered via an infrared laser operating at a wavelength within one of the following ranges: 700-950 nm, 1000-1350 nm, 1600-1870 nm, 2100-2300 nm.

28. The method of claim 15, said irradiation of said nanoparticles being delivered via an external laser emitter generating infrared radiation energy at not more than 5 W, and which: omits a liquid cooling circuit, and operates at less than 30 dB while generating said infrared radiation.

29. The method of claim 15, said irradiation of said nanoparticles being achieved via a laser emitter no part of which physically contacts the kidney stone.

30. The method of claim 15, said nanoparticles being irradiated from a distance of more than 3 mm.

31. The method of claim 15, said photonic nanoparticles comprising a fullerene.

32. The method of claim 31, said fullerene being a PHF.

33. The method of claim 31, said fullerene being a C₆₀ fullerene.

34. The method of claim 31, said fullerene being symmetric with respect to one of the following point groups: Cₛ, Cₙᵥ, Dₙₕ, T_{d}, Oₕ, Iₕ or Kₕ.

35. The method of claim 15, said photonic nanoparticles being functionalized with a ligand that interacts with a predetermined kidney-stone composition, wherein resulting interactions produce adhesion there between.

36. The method of claim 15, said nanoparticles being suspended in a biocompatible gel to yield a nanoparticle suspension, said nanoparticle suspension being delivered to the vicinity of said kidney stone so that said suspension at least partially coats the kidney stone.

37. The method of claim 15, excluding placement of a ureteral stent within a ureter of the patient.

38. A combination comprising a kidney stone with a photonic nanoparticle at a surface of the kidney stone, said photonic nanoparticle being functionalized with a ligand that interacts with a predetermined composition of said kidney stone, thereby producing adhesion between the nanoparticle and the kidney stone.
